(19) United States
(12) Patent Application Publication
(10) Pub. No.: US 2009/0177210 A1
(43) Pub. Date: Jul. 9, 2009

(54) Skin treatment tool

(76) Inventor: Roberto Francesco Durso, Queensland (AU)

Correspondence Address: Martin P. Hoffman;Hoffman Wasson & Gitler, 2461 South Clark Street, Suite 522, Crystal Center 2, Arlington, VA, 22202-3823, US

(21) Appl. No.: 12/227,602

(22) PCT Filed: Feb. 22, 2007
(86) PCT No.: PCT/AU2007/000194

§ 371 Date: Nov. 21, 2008

(30) Foreign Application Priority Data
May 25, 2006 (AU) 2006902820

(51) Int. Cl. A61B 17/50

(52) U.S. Cl. 606/133; 606/131

(57) ABSTRACT

A skin treatment tool having a handgrip comprising a body having at least one convexly curved side and a skin contacting element set within the body and having an exposed edge which is convexly curved along its length and which is located outwardly of the convexly curved side of the body and extends therealong, the edge comprising a dull and blunt non-invasive edge.

### TECHNICAL FIELD

This invention relates to a skin treatment tool and in particular to a hand tool for removing dead skin cells and other materials from the skin to enhance the appearance of the skin.

### BACKGROUND ART

The appearance and health of the skin particularly the skin on the face is important to many people at least in western societies. Skin pores however being usually exposed to the atmosphere are subject to bacteria build up as well as grime which affects the appearance of the skin. Outer skin cells that is skin cells in the epidermis also continually die and this also reduces skin appearance. A large range of cosmetic skin treatment products are available which are applied to skin to improve the appearance and health of the skin. These materials however also tend to building up within the pores of the skin. Another problem encountered particularly with the skin on the face of men is that of ingrown hairs which can cause infections in a skin pore. Men with fast growing facial hair also often find shaving difficult because of the condition of the skin.

Many different methods and devices are available for treatment of the skin for the purposes of removing dead skin cells OT any other materials such as makeup to improve skin appearance. For example pumice, loofahs and other abrasive devices are available and are used by applying the devices to the skin and scrubbing the skin with such devices for cleaning the skin and/or removing skin cells. Whilst these devices are partly effective in this application, they can cause irritation to the skin particularly if the skin is scrubbed excessively. Abrasive lotions can also be applied to the skin however lotions of this type can also cause irritation.

An alternative means for treating the skin is to use laser therapy. Laser therapy procedures however can be relatively expensive and the results of laser therapy are not always effective. None of the above-described procedures necessarily provide long-term benefits.

### SUMMARY OF THE INVENTION

The present invention aims to overcome or alleviate the above disadvantages by providing a skin treatment tool which is of relatively simple construction, which is effective in removing skin cells and other materials from the skin of a person and which may be easily used by a user. Other objects and advantages of the invention will become apparent from the following description.

The present invention thus provides in a first preferred aspect, a skin treatment tool, said tool comprising a handle defining a gripping member for said tool and a sheath formed with said handle, a skin contacting element set within said sheath said element having a dull and blunt operative edge, said sheath sheathing opposite sides of said element and leaving said edge exposed, said exposed edge being convexly curved along its length.

The skin contacting element suitably comprises a thin planar member which may be of blade-like form. The element is suitably squared off by the intersection between the opposite planar sides of the member and end of the member and thus is a non-invasive and not sharp edge. Opposite corners are thus defined in the edge between the opposite planar sides and end of the member, the opposite corners extending along the convex curvature of the edge. Preferably the exposed edge of the skin contacting element has a constant radius at least along a substantial portion of its length The exposed edge may have a constant radius along its full length.

The skin contacting element is preferably made from stainless steel or teflon however other materials may be used such as other plastics.

The handle is suitably of elongated form and symmetrical about a longitudinal axis. The skin contacting element suitably is aligned with the longitudinal axis.

The gripping member of the handle suitably includes non-slip means whereby the handle may be readily gripped. The non-slip means suitably comprises a plurality of locating recesses for locating the fingers of a user.

The handle may include a hole therethrough adjacent its free end whereby the tool may be hung from a hook or the like. The gripping member and sheath of the handle are suitably formed integrally. The handle is suitably moulded of plastics.

The sheath suitably has a concave lower side and tile skin, contacting element suitably extends from the concave side of the sheath. The sheath is suitably provided with a recess, channel or slot on its lower side to receive the element. The recess, channel or slot suitably is of a width which is substantially the same as the width of the skin contacting element such that the skin contacting element may be neatly and firmly received and held within the recess, channel or slot.

The present invention provides in a further preferred aspect a skin treatment tool, said tool having a handgrip comprising a body having opposite faces and at least one convexly curved side, a skin contacting element set within said body and having an exposed edge which is convexly curved along its length, said edge being located adjacent to and positioned outwardly of said convexly curved side of said body and extending therealong, said edge comprising a dull and blunt edge.

Preferably the edge is at a substantially constant distance from the one side of the body along substantially the full length of the one side such that the edge of the element is substantially parallel to the one side of the body. The edge is suitably located close to the one side of the body so as to project only a short distance outwardly of the body. Typically the edge extends from about 1.00 to 3.00 mm. from the one side of the body. The edge most preferably is spaced about 1.5 mm from the body for substantially its full length.

The skin contacting element may be of blade-like form and may suitably comprises a thin substantially planar member having opposite parallel sides and a squared-off end forming the element edge. The opposite sides of the planar member suitably intersect the end of the planar member to define in the exposed edge, opposite corners which extend along the convex curve of the edge. The edge is thus a non-sharp non-invasive edge.

Preferably the body has a recess, channel or slot extending inwardly from the one side in which the element is located. The recess, channel or slot is of substantially the same width as the element such that the element is firmly received therein and supported along its length.

In a particularly preferred form, the body suitably comprises a substantially curvilinear triangular body, the body having convexly curved outer sides, one side of which defines the one side of the body. The curvilinear triangular body may be in the general configuration of an isosceles triangle.

Preferably the body is provided with a cavity on at least one face for locating the fingers or thumb of a user. The cavity is suitably arranged generally centrally of the body. The cavity suitably extends in the same direction as the one side of the body and extends symmetrically relative to a central line of symmetry of the body passing through the one side of the body. The cavity suitably is concave in cross section. The cavity suitably is of generally oval configuration plan view. Preferably cavities of the above type are provided in opposite faces of the body.

The handgrip is suitably moulded of plastics. The skin contacting element may be metal for example stainless steel or plastics such as teflon.

### BRIEF DESCRIPTION OF THE DRAWINGS

In order that the invention may be more readily understood and put into practical effect, reference will now be made to the accompanying drawings which illustrate preferred embodiments of the invention and wherein:

FIG. 1 is a plan view of a skin treatment tool according to one embodiment of the invention;

FIG. 2 is a side view of the tool of FIG. 1;

FIG. 3 is an end view of the tool of FIG. 1;

FIG. 4 is a side view of the skin contacting element of the tool;

FIG. 5 is a perspective view of a skin treatment tool according to a further embodiment of the invention;

FIGS. 6, 7 and 8 respectively arc side, end and plan views of the tool of FIG. 5;

FIG. 9 is a sectional view of the tool along line A-A of FIG. 6;

FIG. 10 is a sectional view of the tool along line B-B of FIG. 7;

FIG. 11 is a sectional view of the tool handgrip showing the mounting slot for tile skin contacting element;

FIG. 12 is a side elevational view of the skin contacting element of the tool of FIG. 5; and

FIG. 13 is an enlarged view of the region C of FIG. 7 showing details of the exposed edge of the skin contacting element.

### DETAILED DESCRIPTION OF THE PREFERRED EMBODIMENT

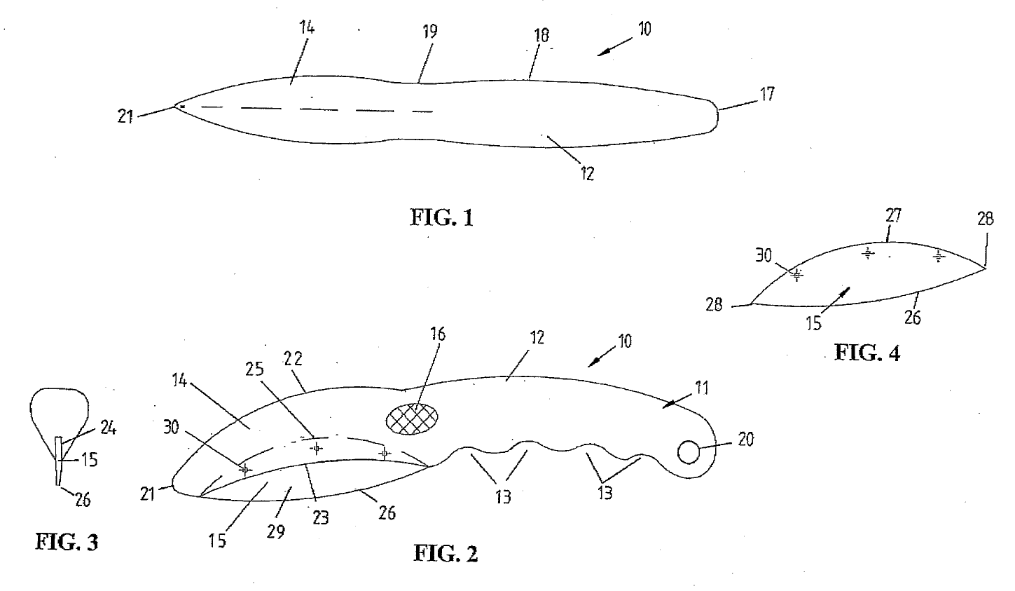

Referring to the drawings and firstly to FIGS. 1 and 2, there is illustrated a skin treatment tool 10 according to a first embodiment of the invention, the tool 10 having a one-piece elongated handle 11 designed to fit most hand sizes, the handle 11 including an elongated gripping member 12 formed on its lower edge with a plurality of finger recesses 13 to receive the four fingers of the user and a sheath 14 for supporting and sheathing opposite sides of a skin contacting element 15 which is of a planar blade-like form. The gripping member 12 also includes on opposite sides thumb rests 16 for locating the thumb of a user when the gripping member 12 is gripped by the right or left hand of the user. The thumb rests 16 may be defined by roughened regions on opposite sides of the member 12.

The gripping member 12 has a minimum width at region 17 at its free end and increases smoothly and symmetrically in width along its length to a maximum at region 18 intermediate the ends of the gripping member and thereafter tapers smoothly in width along the gripping member 12 towards die sheath 14 to a minimum at region 19 at which the thumb rests 16 are located. The gripping member 12 thus comprises an ergonomic design to fit most hand sizes with the taper in the gripping member 12 providing a sound comfortable feeling in the hand. The gripping member 12 additionally includes adjacent its free end a through aperture 20 which enables the tool 10 to be readily hung from a hook or the like for example in a shower or bath.

The sheath 14 comprises a continuation of the gripping member 12 and as shown in FIG. 1 increases slightly in width from the region 19 and then tapers to a point 21 at the end of the tool 10. The sheath 14 is convexly curved at 22 on its top side down to the point 21 and is concavely curved at 23 on its lower side. The tool handle 11 as is apparent in FIG. 3 has a maximum width on or towards its upper side and tapers in width symmetrically to its lower side.

A channel-like recess or slot 24 is formed centrally in the lower concave side 23 along the longitudinal centreline of the sheath 14 to accept the element 15, the recess or slot 24 being of substantially the same width as the element 15 and having a concavely curved inner edge 25.

The element 15 as shown in FIG. 4 includes a convexly curved outer edge 26 and a convexly curved inner edge 27 complementary to the recess edge 25, the edges 26 and 27 extending between opposite points 28. When the element 15 is located in the recess or slot 24 as in FIG. 2, the element 15 has an exposed portion 29 which extends convexly from the sheath 14 between the end points 28. The exposed portion 29 tapers in width at opposite ends into the sheath 14 so that the opposite points 28 are within the sheath 14 whereby a smooth transition is provided between the edge 26 and the sheath 14 at opposite ends of the element 15. This provides the tool 10 with stylish smooth lines and ensures safety as the element points 28 are not exposed. This also enables the element 15 to be easily cleaned.

To secure the element 15 within the sheath 14, the element 15 is provide with three spaced extrusion holes 30 for holding the element 15 in place within the sheath 14. The holes 30 may accept rivets or other fasteners driven through the sheath 14 or the tool handle 11 may be moulded with element 15 in situ so that the sheath 14 is moulded around the element 15. The element 15 may be manufacture from stainless steel or teflon and the edge 26 comprises a substantially blunt and dull edge. Thus whilst the element 15 as shown in FIG. 3 tapers towards the edge 26, it does not taper to a point or line but is rather “squared off”. Thus at the edge 26, two coalers are defined on opposite sides defined by the intersection between the sides of the element 15 and end of the element 15.

In use, the gripping member 12 is gripped by the user and the edge 6 is applied to the skin of the user usually at an angle and moved over the skin in a scraping manner such that it removes dead skin cells and other materials on the skin or in the skin pores and/or hair.

The tool 10 may typically be kept in the shower and hung via the aperture 20 and be typically used during normal showering when the skin is moist.

The element 15 in the embodiment illustrated has an operative edge 26 with a radius of between 120 mm and 170 mm however this radius may vary.

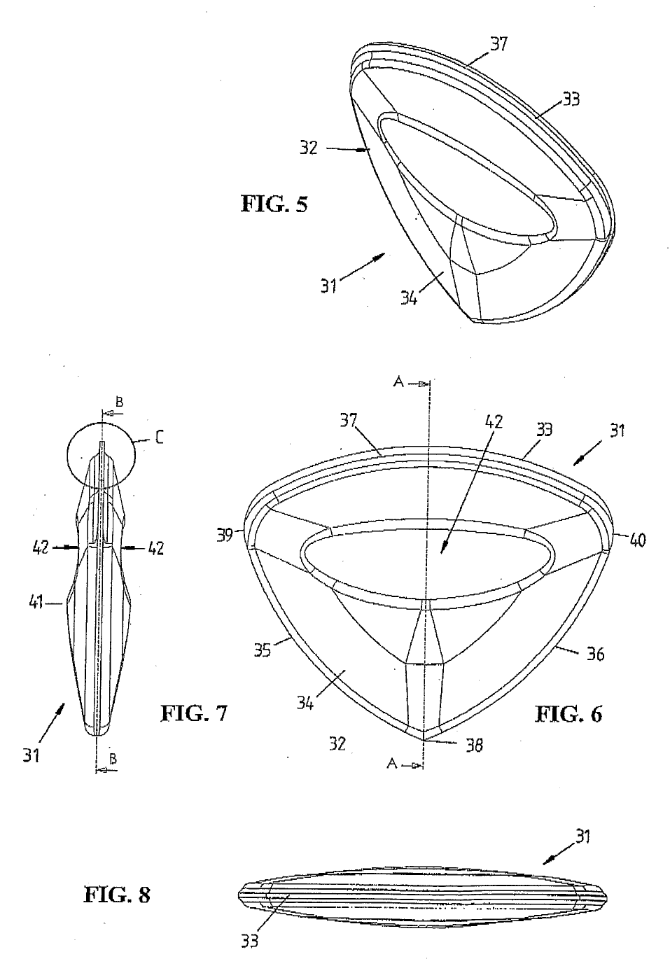

Referring now to FIGS. 5 to 8 there is illustrated a further embodiment of skin treatment tool 31 according to the invention. The tool 31 includes a handgrip or handle 32 and skin contacting, element 33, the handle 32 having a body 34 of a generally curvilinear isosceles triangular configuration in side view and being symmetrical about the section lines A-A and B-B. The body 34 has opposite equal sides 35 and 36 and a third side 37 at which the element 33 is located. Each side 35, 36 and 37 is convexly curved with the equal sides 35 and 36 which have substantially the same constant radius along their length meeting at a pointed corner 38. The third side 37 has a radius greater than the radius of the sides 35 and 36 along its length other than at its opposite ends where the third side 37 meets the equal sides 35 and 36 through convexly curved corners 39 and 40.

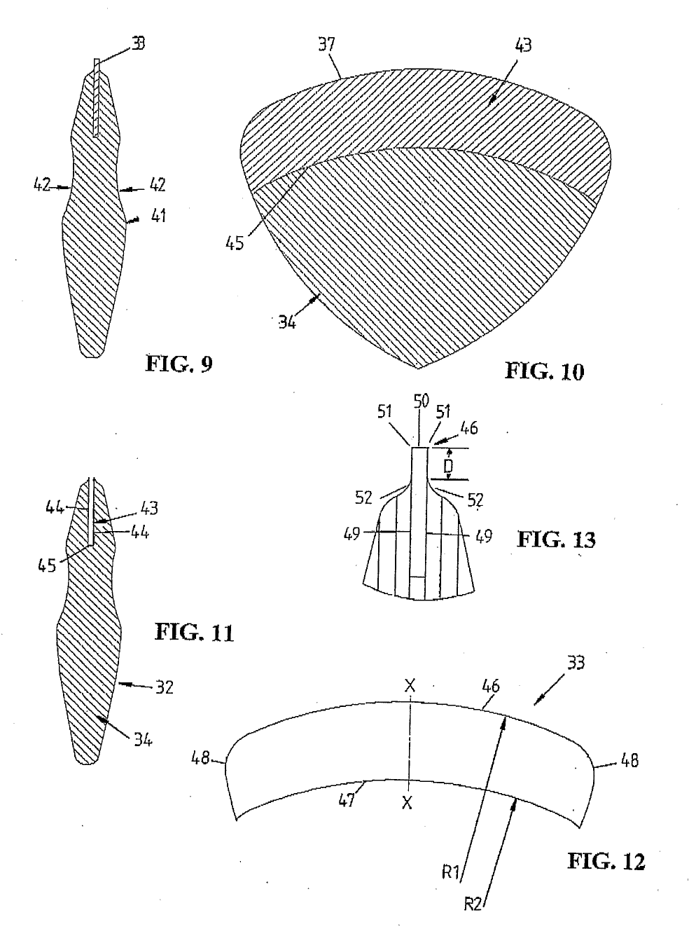

The body 34 of the hand grip 32 has as shown in FIGS. 7 and 9 a maximum width generally centrally of the body 34 as at 41 and tapers in width outwardly in all directions from the maximum thickness region 41 to the sides 35, 36 and 37 and corners 38, 39 and 40 of the body 34. The body 34 has a maximum thickness which enables it to be easily received and gripped in the band. Typically the body 34 has a maximum thickness dimension of between 10 and 20 mm and preferably between approximately 11 mm and 15 mm. The body 34 additionally is provided in its opposite faces with generally oval shaped cavities 42, the cavities 42 being arranged generally centrally of the faces and extending generally parallel to the side 37 of the body 34 and extending symmetrically to opposite sides of the centreline A-A through the body 34. The cavities 42 are concavely curved in cross section and act as locating recess for the fingers and thumb of the hand for gripping the opposite faces of the body 34.

Extending into the body 34 from the side 37 is a thin channel or slot 43 for receiving the element 33 as shown in FIG. 11, the slot 43 lying along the central, plane of the body 34 at line B-B and extending the full length of the side 37 to open through the opposite corners 38 and 39 of the body 34. The slot 43 is defined by parallel side walls 44 and a bottom wall 45 which for a majority of its length has a curvature substantially the same as the curvature of the side 37. The channel or recess 43 has a depth slightly less that the depth of the element 33.

The element 33 as shown in FIG. 12 comprising a thin planar member having an outer operative edge 46 which has a curvature of radius R1 substantially the same as the radius of curvature of the body side 37. The element 33 additionally has an inner edge 47 which has a curvature of radius RN which is substantially the same as radius R1, the radius R2 being along on the same center line X-X of the element 33 but at a different position along the line X-X. The inner edge 47 of the element 33 is thus of substantially the same curvature as the bottom wall 45 of the slot 43 so as to sit against the wall 45 when assembly. Furthermore, the configuration of the element 33 is such that a series of elements 33 can be manufactured from for example sheet metal, one nested with the next element. The distance between the edges 46 and 47 is slightly greater that the depth of the slot 43 and the element 33 has a length substantially the same as the length of the recess 43. The element 33 is also convexly curved from the outer edge 46 to its opposite ends 48.

The edge 46 of the element 33 is blunt and as shown in FIG. 13 is squared off by the right-angle intersection between the opposite planar sides 49 of the element 33 and end 5 of the element 33. This defines an edge 46 with two opposite square corners 51 on opposite sides of the element 33 which extend along the curvature of the edge 46.

The element 33 is located within the slot 43 to be sheathed on opposite sides and so that the edge 46 of the element 33 is exposed and extends parallel to the side 37 of the body 34 substantially along the fill length of the side 37 as shown in FIG. 6 with the edge 46 carving at its opposite ends 48 to merge smoothly into the body 34. The edge 46 projects a short distance “D” outwardly of the side 37 of the body 34 as shown in FIG. 13 so that the corners 51 are exposed. As is apparent in FIGS. 11 and 13, the side 37 of the body 34 on opposite sides of the element 33 is concavely curved as at 52 so as to smoothly merge into the element 33 and ensure that the tool 31 call be used without fouling the side 37 of the body 34.

In use the handgrip 32 of the tool 31 is gripped in the hand with the recesses 42 defining locating means for the thumb and fingers on opposite sides of the tool body 34. The tool 31 is then applied typically at an angle to the skin where skin cells or other materials are to be removed such that one of the corners 51 of the element 33 contacts the skin. Typically this is the skin on the face. By moving the tool 31 over the skin a corner 51 of the element 33 will scrape cells or other materials such as ingrowing hairs from the skin. The blunt configuration of the edge 46 will ensure that the skin is not damaged by the element 33 and will not cause irritation to the skin. This is also facilitated by the convex curvature of the edge 46 of the element 33.

The tool 31 may be constructed by moulding the, body 34 for example of plastics and thereafter the element 33 may be inserted into the slot 43 and secured therein by adhesives or alternatively by rivets or other fasteners. Alternatively the body 34 may be moulded about the element 33.

The tool 31 may be of various sizes however it has been found that the tool is particularly effective where the outer edge 46 of the element 33 of the tool 31 and thus the outer side 37 of the body 34 has a radius of between 60 mm and 100 mm. In particularly preferred embodiments, the edge 46 has a radius of 62 mm and 92 mm where the body 34 has a maximum width of 70 mm and 90 mm respectively between corners 39 and 40. The body 34 however may have a reduced maximum width such as 50 mm and the radius of the side 37 is correspondingly reduced. In the specific sizes referred to above, the maximum thickness of the body is typically between 10 mm and 15 mm and thus is about 15 to 20% of the width.

The element 33 also has a preferred thickness of between 0.5 mm and 1.5 mm with a particularly preferred width being 0.9 mm. The edge 46 typically projects a distance “D” of 1.0 mm to 3.0 mm outwardly from the side 37 the body 34 of the handgrip 31 with the most preferred distance being 1.5 mm.

The tools of the invention can help prevent all types of skin problems such as acne, blackheads, flaking and ingrown hair irritation. It has been found that the skin after treatment is particularly smooth and thus this makes the skin more readily suitable for the application of skin treatment products. In the case of use of the tool on a man's face, subsequent shaving procedures using blades or electric shavers will prove more effective because of the prior removal of dead skin, ingrowing hairs or other skin deposits. When used in conjunction with the application of water, dead skin cells and impurities are washed away and not left on the pillow or bed which is particularly useful for people who suffer from breathing problems associated with dust mites.

Whilst the embodiments describe tools of particular configurations, it will be appreciated that the design thereof can considerably vary from that illustrated and described.

The terms “comprising” or “comprises” as used throughout the specification and claims are taken to specify the presence of the stated features, integers and components referred to but not preclude the presence or addition of one or more other feature/s, integer/s, component/s or group thereof.

Whilst the above has been given by way of illustrative embodiment of the invention, all such variations and modifications thereto as would be apparent to persons skilled in the art are deemed to fall within the broad scope and ambit of the invention as defined in the appended claims.

1-18. (canceled)

19. A skin treatment tool for application to the skin for scraping dead skin cells or hairs from the skin, said tool comprising a body having opposite faces and at least one side which is convexly curved, a skin contacting element comprising a substantially planar member, said planar member having opposite sides and having an edge which is convexly curved along the length of said element, a recess, channel or slot extending inwardly from the one side of the body, said element being received within said recess, channel or slot with said edge being located outwardly of said convexly curved side of said body and extending therealong, said recess, channel or slot being of substantially the same width as the element such that the element is supported along its length, said edge comprising a dull and blunt edge.

20. A skin treatment tool as claimed in claim 19 wherein said edge is defined by an end of said element and wherein the intersection of said opposite sides of said planar member with said end defines in the exposed edge, opposite corners which extend along the convex curve of the edge.

21. A tool as claimed in claim 20 wherein said body is provided with a cavity on at least one said face for locating the fingers or thumb of a user, said cavity being concave in cross section and extending in the same direction as said one side and said edge.

22. A tool as claimed in claim 19 wherein said exposed edge has a radius of between 60 mm and 100 mm.

23. A tool as claimed in claim 19 wherein said edge extends between 1 mm and 3 mm outwardly from said one side.

24. A skin treatment tool for application to the skin for removing dead skin or hairs from the skin, said skin treatment tool having a handgrip comprising a body having opposite faces and a convexly curved outer side, a skin contacting element set within said body and having an exposed edge which is convexly curved along its length, said exposed edge being located outwardly of said convexly curved side of said body and extending therealong at a substantially constant distance from the one side of the body along substantially the full length of the one side such that the edge of said element is substantially parallel to the one side of the body, said edge comprising a dull and blunt edge.

25. A tool as claimed in claim 24 wherein said edge is at a substantially constant distance from the convexly curved side of the body along substantially the full length of the side such that the edge of said element is substantially parallel to the one side of the body.

26. A tool as claimed in claim 24 wherein said element comprises a substantially planar member having opposite sides and wherein said opposite sides of the planar member form in said exposed edge, opposite square corners.

27. A tool as claimed in claim 24 wherein said body has a recess, channel or slot extending inwardly from the one side in which the element is neatly located and wherein said recess, channel or slot is of substantially the same width as the element such that the element is supported along its length.

28. A tool as claimed in any claim 24 wherein said body comprises a substantially curvilinear triangular body, the body having three convexly curved outer sides, and wherein said exposed edge extends along one said side.

29. A tool as claimed in claim 28 wherein said body is provided with a cavity in at least one said face for locating the fingers or thumb of a user, said cavity being arranged substantially centrally of the body and extending in the same direction as the one side of the body.

30. A tool as claimed in claim 29 wherein respective said cavities are provided in opposite faces of the body.

31. A tool as claimed in claim 24 wherein said exposed edge has a radius of between 60 mm and 100 mm.

32. A skin treatment tool for application to the skin for scraping dead skin or hairs from the skin, said skin treatment tool having a handgrip comprising a substantially curvilinear triangular body, the body having opposite faces and three convexly curved outer sides, a skin contacting element set within said body and having an exposed edge which is convexly curved along its length, the edge of said element being located outwardly of one said convexly curved side of said body and extending therealong at a substantially constant distance from the one side of the body along substantially the full length of the one side such that the edge of said element is substantially parallel to the one side of the body, said edge comprising a dull and blunt edge, and a cavity in at least one face of the body for locating the fingers or thumb of a user.

33. A tool as claimed in claim 32 wherein said body has a recess, channel or slot extending inwardly from the one side, said element being located within said recess, channel or slot and being of substantially the same width as the element such that the element is supported along its length.

34. A tool as claimed in claim 32 wherein said element has opposite ends which curve convexly from said exposed edge into said body.

35. A tool as claimed in claim 32 wherein respective said cavities are provided in opposite faces of the body.

36. A tool as claimed in claim 35 wherein said cavities extend in the direction of said element.

37. A tool as claimed in claim 32 wherein said exposed edge has a radius of between 60 mm and 100 mm.
